(19) United States
(12) Patent Application Publication
(10) Pub. No.: US 2026/0188519 A1
(43) Pub. Date: Jul. 2, 2026

(54) INFORMATION PROCESSING DEVICE, INFORMATION PROCESSING METHOD, AND COMPUTER PROGRAM

(71) Applicant: PEPETUAL STREAM LLC, Tokyo (JP)

(72) Inventors: Shintaro OYAMA, Aichi (JP); Tamaki KINOSHITA, Aichi (JP); Hiroshi ARIMA, Aichi (JP); Daisuke HAGIWARA, Aichi (JP)

(73) Assignee: PEPETUAL STREAM LLC, Tokyo (JP)

(21) Appl. No.: 18/862,561

(22) PCT Filed: Apr. 26, 2023
(86) PCT No.: PCT/JP2023/016397

§ 371 Date: Sep. 2, 2025

(30) Foreign Application Priority Data
May 12, 2022 (JP) 2022-078809

(51) Int. Cl. G16H 50/50; G16H 10/60; G16H 50/30

(52) U.S. Cl. CPC G16H 50/50; G16H 10/60; G16H 50/30

(57) ABSTRACT

An information processing device for predicting a serum sodium concentration at a future time has a predictor acquisition unit and a prediction execution unit. The predictor acquisition unit acquires a target person's predictor to be predicted; the predictor includes a measured value of the serum sodium concentration at a reference time and an index value indicating the sodium intake in a future period, which is a period from the reference time to the future time. The prediction execution unit inputs the target person's acquired predictor into a serum sodium concentration prediction model created by machine learning using training data, where the predictor and a measured value of the serum sodium concentration at the future time are associated with each other, and predicts the target person's serum sodium concentration at the future time.

### TECHNICAL FIELD

The technology disclosed in the present specification relates to an information processing device for predicting a serum sodium concentration at a future time, and the like.

### BACKGROUND ART

Hyponatremia is an electrolyte disorder in the body when the serum sodium concentration is below a predetermined level (e.g., 135 mEq/L), and is accompanied by symptoms such as nausea, headache, and light-headedness. Severe hyponatremia (e.g., the serum sodium concentration being below 125 mEq/L) can cause serious Central Nervous System symptoms, including impaired consciousness, spasms, and a coma.

Hyponatremia is generally classified into three categories: (1) symptoms associated with a decrease in ECFV (Extracellular Fluid Volume), (2) symptoms with a nearly normal ECFV, and (3) symptoms associated with an increase in ECFV. Treat hyponatremia associated with decreased ECFV loss includes administration of infusions (e.g., physiological saline, hypertonic (3%), saline, and No. 3 solution (% glucose and electrolyte solution), glucose solution) to replenish ECF (Extracellular Fluid). In contrast, treat hyponatremia with near-normal or increased ECFV includes water restriction and administration of vasopressin V2 receptor antagonists to promote excretion of free water in the body. Thus, the treatments for the two are polar opposites. Therefore, it is important to accurately understand the pathology of each case to differentiate the hyponatremic condition in each case, and to provide appropriate treatment according to each pathology. However, it is extremely difficult to accurately assess the ECFV, and it is therefore difficult to ascertain the disease's pathology and to differentiate the hyponatremic condition based on the ECFV before starting treatment. There are also cases with multiple coexisting conditions and cases where the condition changes between the early stages of the disease and during treatment.

In the treat hyponatremia, an excessive rapid increase in serum sodium concentration (e.g., by 8-10 mEq/L/day or more) may cause Osmotic Demyelination Syndrome (ODS). The ODS causes serious symptoms, such as dysarthria, dysphagia, quadriplegia, and disturbances of consciousness, and is often fatal. There is no treatment with established efficacy. Therefore, it is crucial for a patient to avoid a rapid increase in serum sodium concentration in the treat hyponatremia to prevent ODS.

As described above, however, in clinical practice, it is difficult to accurately identify this disease's condition before starting treatment and to differentiate hyponatremia, and moreover, the disease's condition may change during treatment. Therefore, even when serum sodium concentrations are frequently monitored in target patients under treatment, a rapid increase in serum sodium concentrations may occur. In addition, there are medical institutions where it is difficult to conduct frequent blood tests at night.

Conventionally, a system for predicting various medical events based on information in a patient's electronic medical record has been proposed (for example, Patent Document 1).

### PRIOR ART DOCUMENTS

### Patent Documents

Patent Document 1: Japanese Patent Application Publication No. 2020-529057

### SUMMARY OF THE INVENTION

### Problem to be Solved by the Invention

The conventional system, however, does not predict the serum sodium concentration at a future time. In order to appropriately perform the treat hyponatremia, a technique for accurately predicting the serum sodium concentration at a future time is desired.

These issues are not limited to predicting the serum sodium concentrations at a future time in target patients being treated for hyponatremia. In an athlete, such as a long-distance runner, when oral supplementation is given, a similar issue exists in predicting the serum sodium concentrations at a future time. The issue is that a technique for accurately predicting the serum sodium concentration at a future time for all humans is currently unknown.

The present specification discloses techniques capable of solving the above-described issue.

### Solution to the Problem

The technique disclosed in the present specification can be realized, for example, in the following embodiments.

(1) An information processing device disclosed in the present embodiment for predicting a serum sodium concentration at a future time has a predictor acquisition unit and a prediction execution unit. The predictor acquisition unit acquires a target person's predictor to be predicted; the predictor includes a measured value of the serum sodium concentration at a reference time and the index value indicating the sodium intake in a future period, which is a period from the reference time to a future time. The prediction execution unit inputs the predictor acquired from the target person into a serum sodium concentration prediction model created by machine learning using training data where the predictor and a measured value of the serum sodium concentration at the future time are associated with each other, and it predicts the target person's serum sodium concentration at the future time.

As described above, in the present information processing device, the target person's serum sodium concentration at a future time can be predicted with high accuracy by inputting into the serum sodium concentration prediction model a predictor, including the measured value of the target person's serum sodium concentration for prediction at the reference time and the index value indicating the target person's sodium intake in the future period for prediction.

(2) The above information processing device may further have a prediction result output unit which outputs a prediction result of a target person's serum sodium concentration. According to the present configuration, the user of the device can recognize the predicted value of the target person's serum sodium concentration at a future time.

(3) The above information processing device may further have a training data acquisition unit that acquires the training data; and a model acquisition unit that creates the serum sodium concentration prediction model by machine learning using the training data. According to the present configuration, the serum sodium concentration prediction model can be generated without using any other device, and the target person's serum sodium concentration at a future time can be predicted using the model.

(4) The above information processing device may further have a model updating unit which updates the serum sodium concentration prediction model by machine learning using the updated training data, including data where the target person's predictors and the measured value of the serum sodium concentration at the future time are associated with each other. According to the present configuration, the serum sodium concentration prediction model can be converted into a model more suitable for the target person's characteristics, and the prediction accuracy of the serum sodium concentration can be improved.

(5) In the above information processing device, the predictor acquisition unit acquires information specifying a type of the intake item to be taken by the target person, and also acquires an index value indicating the sodium intake in a future period by referring to information indicating the sodium content contained in each of the intake items. According to the present configuration, it is possible to acquire an index value representing the sodium intake in the future period only by the user designating the type of the intake item, and it is possible to more efficiently predict the serum sodium concentration.

(6) In the above information processing device, the predictors may include an index value representing water intake in the future period; and an index value representing a water discharge amount in a past period, which is a period from a past time to the reference time. According to the present configuration, the accuracy of predicting the serum sodium concentration can be improved.

(7) In the above information processing device, the target person may be a patient with hyponatremia. According to the present configuration, it is possible to predict the serum sodium concentration when a predetermined treatment is given to a patient with hyponatremia.

(8) The above information processing device may further have a notification unit for providing a given notification when the rate of increase in the concentration changes from the measured value of the target person's serum sodium concentration at the reference time to the predicted value of the target person's serum sodium concentration at the future time. According to the present configuration, it is possible to prevent the rate of increase in the serum sodium concentration from becoming excessively fast, during the treatment of a patient with hyponatremia, and it is possible to avoid the generation of ODS, for example.

The technology disclosed in the present specification can be realized in various forms, for example, an information processing device, an information processing method, a computer program based on the method, and a non-temporary recording medium on which the computer program is recorded.

### BRIEF DESCRIPTION OF DRAWINGS

FIG. 1 is an explanatory diagram that conceptually illustrates a serum sodium concentration prediction model MO.

FIG. 2 is an explanatory diagram that illustrates the configuration of the hospital system 10.

FIG. 3 is a block diagram that conceptually illustrates the configuration of the terminal device 100.

FIG. 4 is an explanatory diagram that illustrates an example of the infusion information ID.

FIG. 5 is a flowchart that illustrates a serum sodium concentration prediction model acquisition process.

FIG. 6 is a flowchart that illustrates a serum sodium concentration prediction process.

FIG. 7 is an explanatory diagram that illustrates the prediction accuracy of a serum sodium concentration prediction model MO of the present example.

FIG. 8 is an explanatory diagram that illustrates the prediction accuracy of a serum sodium concentration prediction model MO when a learning model and a predictor are changed in various ways.

### DESCRIPTION OF THE EMBODIMENTS

### A. Present Embodiment

### A-1. Outline of a Serum Sodium Concentration Prediction Model MO

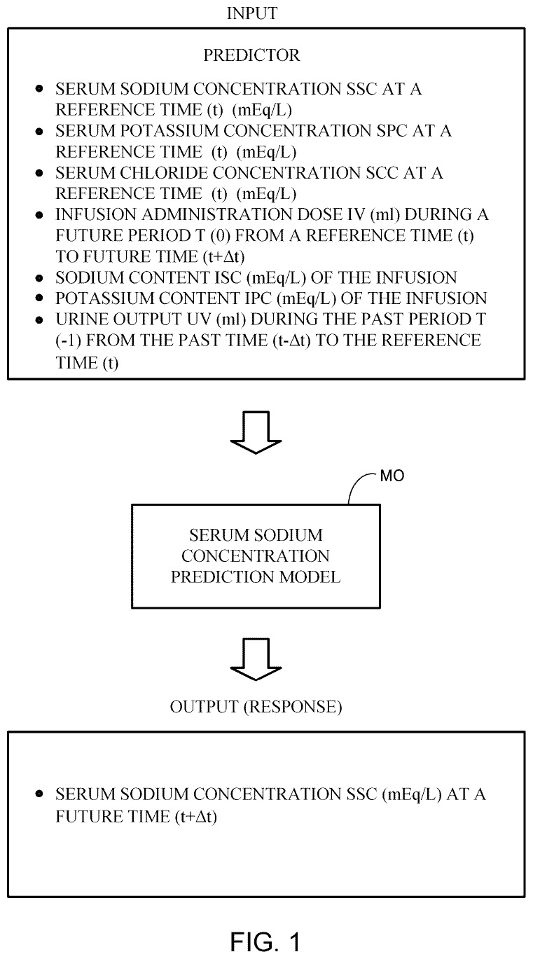

First, the outline of the serum sodium concentration prediction model MO in the present embodiment will be described. FIG. 1 is the explanatory diagram that conceptually illustrates a serum sodium concentration prediction model MO.

The serum sodium concentration prediction model MO is a learned model used to predict a serum sodium concentration at a future time. As shown in FIG. 1, the serum sodium concentration prediction model MO is a machine learning model that takes seven predictors as input and the serum sodium concentration SSC (t+Δt) (mEq/L) at a future time (t+Δt) as output (response). In the present embodiment, the input predictors include the following seven items.
Measured value (mEq/L) of serum sodium concentration SSC at a reference time (t)
Measured value (mEq/L) of serum potassium concentration SPC at a reference time (t)
Measured value (mEq/L) of serum chloride concentration SCC at a reference time (t)
Infusion administration dose IV (ml) during a future period T(0) from a reference time (t) to a future time (t+Δt)
Sodium content ISC (mEq/L) of the infusion
Potassium content IPC (mEq/L) of the infusion
Urine output UV (ml) during the past period T(−1) from the past time (t−Δt) to the reference time (t)

The reference time (t) is a time serving as a reference for predicting the serum sodium concentration SSC (t+Δt) at a future time (t+Δt), and is a time when the measured value of the serum sodium concentration SSC (t) is known. The reference time (t) may be any time, and is, for example, a time when the serum sodium concentration is to be predicted using the serum sodium concentration prediction model MO. The time interval between the reference time (t) and a future time (t+Δt) and time (Δt) that indicates the time interval between the past time (t−Δt) and the reference time (t), can take any value in this embodiment. The time interval in the present embodiment is set to a fixed value of 6 hours. The Δt may be a variable value. The future time (t+Δt) may be a future time viewed from the reference time (t), and is not necessarily limited to an actual “future” time. For example, when a time that is shifted back by (ΔtX2) from the present is set as the reference time (t), a time that is shifted back by (t+Δt) corresponds to “future time (t+Δt)” as viewed from the reference time (t).

Among the seven predictors, “the infusion administration dose IV in the future period T(0) from the reference time (t) to the future time (t+Δt)” and/or “the amount of sodium contained in the infusion ISC” can be said to be an index value representing the sodium intake in the future period T(0). The “infusion dose IV in the future period T(0)” can be said to be an index value representing the water intake in the future period T(0). The “urine volume UV in the past period T(−1) from the past time (t−Δt) to the reference time (t)” can be said to be an index value representing the amount of water discharged in the past period T(−1). In the present embodiment, therefore, the predictors as the inputs of the serum sodium concentration prediction model (MO) include: a measured value of the serum sodium concentration at a reference time (t); an index value representing the sodium intake in a future period (T(0)) from the reference time (t) to a future time (t+Δt); an index value representing the water intake amount in the future period (T(0)); and an index value representing the water discharge amount in a past period (T(−1)) from the past time (t−Δt) to the reference time (t).

### A-2. Configuration of Hospital System 10

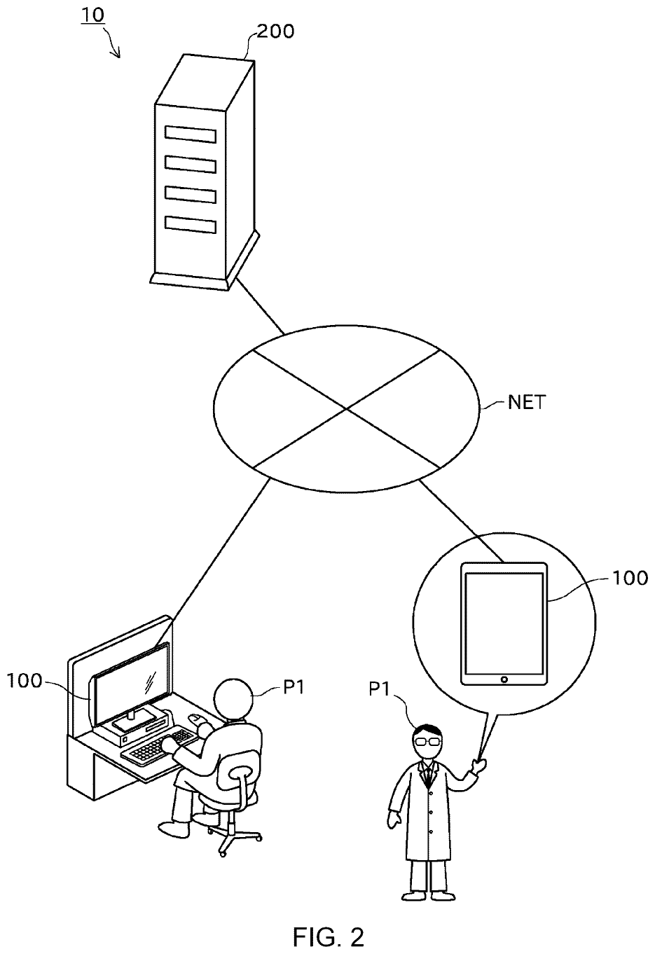

Next, the configuration of the hospital system 10 will be described. FIG. 2 is an explanatory diagram that illustrates the configuration of the hospital system 10. The hospital system 10 is an information system introduced in the hospital. In the hospital system 10 of the present embodiment, the serum sodium concentration prediction model MO is used for a target patient with hyponatremia to predict the serum sodium concentration at a future time when the patient is given a predetermined treatment.

As shown in FIG. 2, the hospital system 10 has a terminal device 100 used by a medical worker P1, such as a doctor or a nurse, and a server 200 installed in the hospital. The respective devices included in the hospital system 10 are connected to each other through a communication network NET enabling them to communicate with each other.

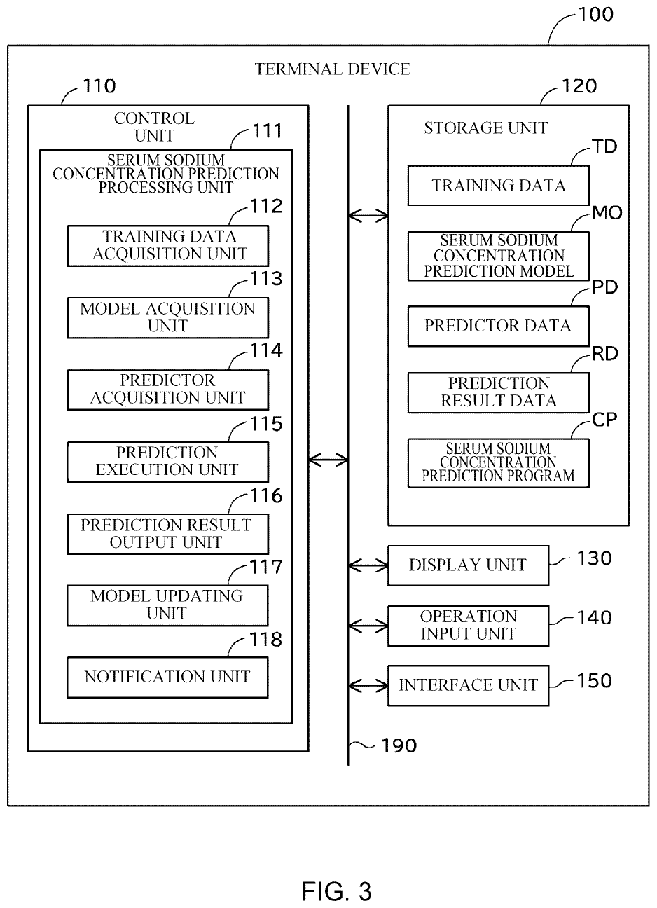

The terminal device 100 is, for example, a PC, a tablet terminal, a smartphone, or the like. FIG. 3 is a block diagram schematically showing the configuration of the terminal device 100. The terminal device 100 has a control unit 110, a storage unit 120, a display unit 130, an operation input unit 140, and an interface unit 150. These units are connected to each other through a bus 190 enabling them to communicate with each other. The terminal device 100 is an example of an information processing device in the claims in the present invention.

The display unit 130 of the terminal device 100 is configured by, for example, a liquid crystal display or the like, and displays various images and information. The operation input unit 140 is configured by, for example, a keyboard, a mouse, buttons, a microphone, or the like, and receives an operation or an instruction of the medical worker P1. The display unit 130 may include a touch panel to function as the operation input unit 140. The interface unit 150 is configured by, for example, a LAN interface or a USB interface, and communicates with other devices connected by wires or wirelessly.

The storage unit 120 of the terminal device 100 is configured by, for example, a ROM, a RAM, a hard disk drive (HDD), a solid state drive (SSD), or the like, and stores various programs and data, and is used as a work area for executing various programs and a temporary storage area for data. For example, the storage unit 120 stores a serum sodium concentration prediction program CP, which is a computer program for executing various processes described later. The serum sodium concentration prediction program CP is provided either in a state of being stored in a computer-readable recording medium (not shown), such as a CD-ROM, a DVD-ROM, or a USB memory, or in a state of being obtainable from an external device (for example, a server on a cloud or another terminal device) via the interface unit 150, and is stored in the storage unit 120 in a state of being operable on the terminal device 100.

In the storage unit 120 of the terminal device 100, training data TD, a serum sodium concentration prediction model MO, predictor data PD, and prediction result data RD are stored in various processes described later. These data and models are described in conjunction with the description of various processes described later.

The control unit 110 of the terminal device 100 is configured by, for example, a CPU or the like, and controls the operation of the terminal device 100 by executing a computer program read from the storage unit 120. For example, the control unit 110 functions as the serum sodium concentration prediction processing unit 111 that executes various processes described later by reading and executing the serum sodium concentration prediction program CP from the storage unit 120. Serum sodium concentration prediction processing unit 111 includes a training data acquisition unit 112, a model acquisition unit 113, a predictor acquisition unit 114, a prediction execution unit 115, a prediction result output unit 116, a model updating unit 117, and a notification unit 118. The functions of these units are described in conjunction with the description of various processes described later.

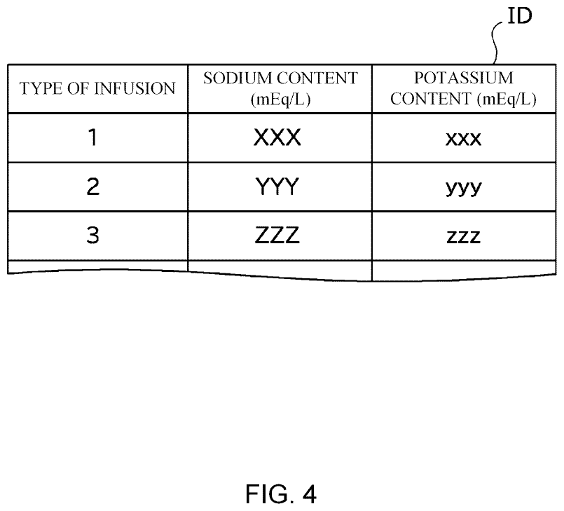

The server 200 (FIG. 2) is a device that provides an information presentation function and an information processing function in the hospital system 10. In the present embodiment, the server 200 stores the infusion information ID. FIG. 4 is an explanatory diagram showing an example of the infusion information ID. The infusion information ID is information that associates the type (preparation) of the infusion administered to the patient with hyponatremia with the amount (mEq/L) of sodium and potassium contained in each type of infusion. By referring to the infusion information ID, the amount of sodium and potassium contained in each type of infusion can be specified.

### A-3. Serum Sodium Concentration Prediction Model Acquisition Process

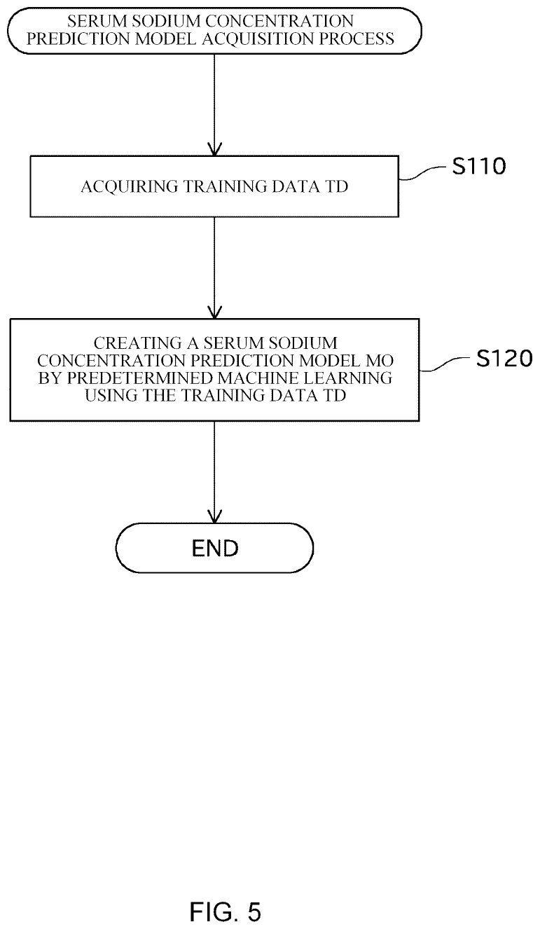

Next, the serum sodium concentration prediction model acquisition process executed by the terminal device 100 of the present embodiment will be described. FIG. 5 is a flowchart that illustrates a serum sodium concentration prediction model acquisition process. The serum sodium concentration prediction model acquisition process is a process for acquiring the above-described serum sodium concentration prediction model MO. In the present embodiment, the terminal device 100 creates the serum sodium concentration prediction model MO by itself by predetermined machine learning, thereby acquiring the serum sodium concentration prediction model MO. The serum sodium concentration prediction model acquisition process is started in response to the medical worker P1 operating the operation input unit 140 of the terminal device 100 to input a start instruction.

First, the training data acquisition unit 112 (FIG. 3) of the terminal device 100 acquires training data TD (S110). The training data TD is a set of a plurality of data where the seven predictors shown in FIG. 1 and the measured value (actual value) of the serum sodium concentration SSC (t+Δt) at a future time (t+Δt) are associated with each other. The training data TD is acquired through the interface unit 150 and stored in the storage unit 120.

Next, the model acquisition unit 113 (FIG. 3) of the terminal device 100 creates a serum sodium concentration prediction model MO by predetermined machine learning (including deep learning) using the training data TD (S120). A model acquisition unit (113) creates a serum sodium concentration prediction model MO by executing machine learning based on a predetermined learning algorithm while referring to predetermined evaluation indices (for example, a root mean square error (RMSE), a mean absolute error (MAE), and a coefficient of determination (R2)), using the seven predictors contained in the training data (TD) as explanatory variables and using a measurement value of a serum sodium concentration SSC (t+Δt) at a future time (t+Δt) contained in the training data TD as an objective variable. Various known learning algorithms can be used for creating the serum sodium concentration prediction model MO; for example, support vector regression, linear regression, decision tree, neural network (including deep neural network), and the like can all be used. The created serum sodium concentration prediction model MO is stored in the storage unit 120 of the terminal device 100. The serum sodium concentration prediction model acquisition process is completed by the above steps.

### A-4. Serum Sodium Concentration Prediction Process

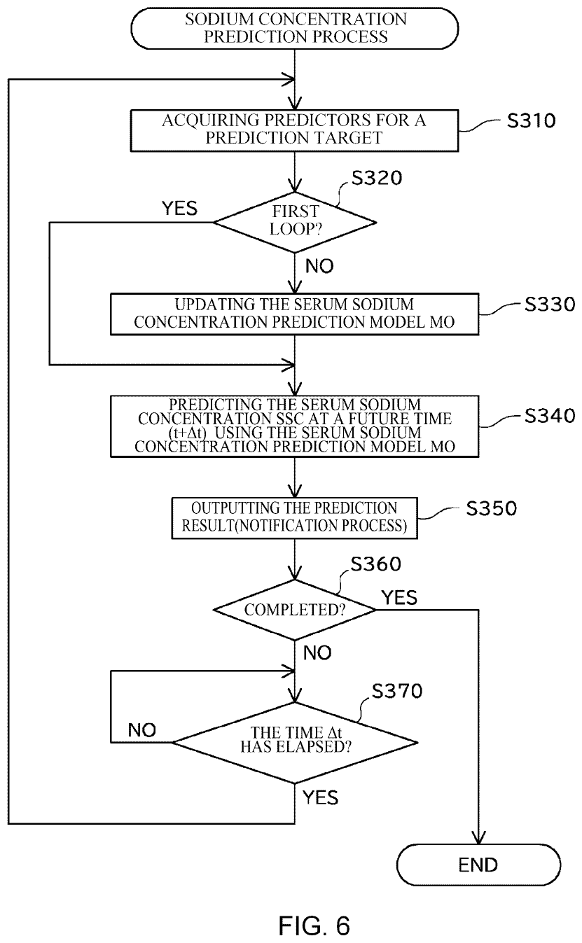

Next, the serum sodium concentration prediction process executed by the terminal device 100 of the present embodiment will be described. FIG. 6 is a flowchart that illustrates a serum sodium concentration prediction process. The serum sodium concentration prediction process is a process for predicting a serum sodium concentration at a future time. Specifically, the serum sodium concentration prediction process is a process for predicting the serum sodium concentration SSC (t+Δt) at a future time (t+Δt) when a predetermined treatment is given to a target patient with hyponatremia by using the serum sodium concentration prediction model MO. The serum sodium concentration prediction process is started in response to operating the operation input unit 140 of the terminal device 100 to input a start instruction of the medical worker P1.

First, the predictor acquisition unit 114 (FIG. 3) of the terminal device 100 acquires the above-described seven predictors (FIG. 1) for the hyponatremic patient who is the prediction target (S310). The acquired predictor is stored in the storage unit 120 as predictor data PD.

Specifically, for the following three predictors among the seven predictors, the medical worker P1 performs a blood test on the target patient and inputs the test results (each measured value) through the operation input unit 140. The predictor acquisition unit 114 acquires each input measurement value. Additionally, these predictors may be acquired by other means (e.g., by means of PoCT or an implantable device). As the predictors, values estimated from the results of analysis of body fluids (e.g., tears and sweat) may be acquired.
Measured value (mEq/L) of serum sodium concentration SSC (t) at a reference time (t)
Measured value (mEq/L) of serum potassium concentration SPC (t) at a reference time (t)
Measured value (mEq/L) of serum chloride concentration SCC (t) at a reference time (t)

For the following three predictors, the medical worker P1 determines the type and dose of the infusion to be administered to the patient, and inputs the determination result (the type and dose of the infusion) through the operation input unit 140. The predictor acquisition unit 114 acquires information for specifying the type and dosage of the input infusion, and acquires the sodium content ISC (mEq/L) and the potassium content IPC (mEq/L) contained in the infusion with reference to the infusion information ID (FIG. 4) stored in the server 200.
Infusion administration dose IV (ml) during a future period T(0) from a reference time (t) to a future time (t+Δt)
Sodium content ISC (mEq/L) of the infusion
Potassium content IPC (mEq/L) of the infusion For the following single predictor, the medical worker P1 measures the patient's urine volume UV in the past period T(−1) and inputs the measurement result (urine volume UV) through the operation input unit 140. The predictor acquisition unit 114 acquires the input urine volume UV.
Urine output UV (ml) during the past period T(−1) from the past time (t−Δt) to the reference time (t) The predictor acquisition unit 114 may acquire the predictor from an electronic medical record provided in the hospital system 10 or an external network.

The serum sodium concentration prediction processing unit 111 (FIG. 3) of the terminal device 100 determines whether or not the loop is the first loop for the target patient to be predicted (S320), and when the loop is the first loop (S320: YES), the serum sodium concentration prediction processing unit 111 skips the process of S330 and proceeds to the process of S340.

Next, the prediction execution unit 115 (FIG. 3) of the terminal device 100 inputs the predictor acquired for the patient to be predicted to the serum sodium concentration prediction model MO, thereby predicting the target patient's serum sodium concentration SSC (t+Δt) to be predicted at a future time (t+Δt) (S340). The prediction execution unit 115 generates a prediction result data RD, which is information indicating a prediction result of the target patient's serum sodium concentration SSC (t+Δt) at a future time (t+Δt), and stores the prediction result data RD in the storage unit 120 of the terminal device 100. The prediction result output unit 116 of the terminal device 100 outputs the prediction result of the target patient's serum sodium concentration SSC (t+Δt) at the future time (t+Δt) based on the prediction result data RD (S350). For example, the prediction result output unit 116 displays the prediction result on the display unit 130. Therefore, the medical worker P1 can recognize the predicted value of the serum sodium concentration SSC (t+Δt) at a future time (t+Δt) when a specific type of infusion is administered to the target patient at a specific dose.

In the present embodiment, the notification unit 118 (FIG. 3) of the terminal device 100 performs a notification process to execute a given notification when the predicted rate of increase of the target patient's serum sodium concentration (that is, the rate of increase of the serum sodium concentration from the measured value of the serum sodium concentration SSC (t) at the reference time (t) to the predicted value of the serum sodium concentration SSC (t+Δt) at a future time (t+Δt)) is faster than a preset threshold value (upper limit value) (S350). The threshold value of the rate of increase in the serum sodium concentration at this time is set to, for example, 8 to 10 mEq/L/day in order to avoid ODS (osmotic demyelination syndrome). The notification process may be performed by, for example, displaying an alarm image by the display unit 130 or outputting an alarm sound by a sound output unit (not shown). By such notification process, the medical worker P1 can recognize that the predicted rate of increase of the serum sodium concentration is excessively fast and may cause ODS.

When such notification process is performed, for example, the medical worker P1 changes the type and/or the dose of the infusion to be administered to the target patient so that the predicted rate of increase in the serum sodium concentration becomes slow, and causes the terminal device 100 to execute the process of S310 to S350 again. By repeatedly performing such a process, the medical worker P1 can determine the type and/or dosage of the fluid so that hyponatremia can be treated as soon as possible while avoiding the occurrence of ODS.

Thereafter, when the serum sodium concentration prediction process is not completed (S360: NO) and the time Δt has elapsed (S370: YES), the serum sodium concentration prediction processing unit 111 of the terminal device 100 similarly executes the process of S310 and subsequent steps described above. In the second and subsequent loops, the reference time (t) is updated to the future time (t+Δt) in the previous loop. Additionally, for example, in S310, the predictor acquisition unit 114 (FIG. 3) acquires the following seven predictors.
Measured value (mEq/L) of serum sodium concentration SSC (t) at a reference time (t) (the future time (t+Δt) in the previous loop)
Measured value (mEq/L) of serum potassium concentration SPC (t) at a reference time (t) (same)
Measured value (mEq/L) of serum chloride concentration SCC (t) at a reference time (t) (same)
Infusion administration dose IV (ml) during a future period T(0) (a further future period following the future period T(0) in the previous loop)
Sodium content ISC (mEq/L) of the infusion
Potassium content IPC (mEq/L) of the infusion
Urine volume UV (ml) during the past period T(−1) (the future period T(0) in the previous loop)

In the second and subsequent loops (S320: NO), the model updating unit 117 (FIG. 3) of the terminal device 100 updates the serum sodium concentration prediction model MO by machine learning using the training data TD, including data where the predictor in the previous loop and the measured value of the serum sodium concentration SSC (t) at the reference time (t) (the future time (t+Δt) in the previous loop) in the current loop are associated with each other (S330). Thus, the serum sodium concentration prediction model MO becomes a model more suitable for the target patient's characteristics (disease state, constitution, etc.), and the prediction accuracy of the serum sodium concentration is improved.

When the end instruction of the serum sodium concentration prediction process is given while the above process is repeatedly executed (S360: NO), the serum sodium concentration prediction process is ended.

### A-5. Examples

An example of the above-described serum sodium concentration prediction model MO will be described below. In the present example, the serum sodium concentration prediction model MO is created by machine learning using the training data TD obtained during the course of treatment of 16 patients with hyponatremia who were hospitalized in the Department of Diabetes and Endocrinology, Nagoya University Hospital.

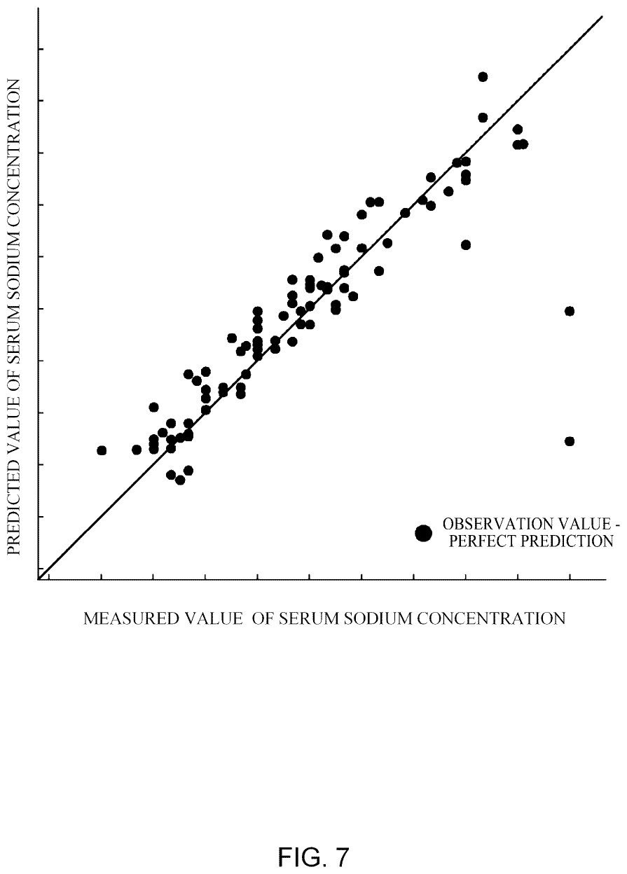

FIG. 7 is an explanatory diagram that illustrates the prediction accuracy of the serum sodium concentration prediction model MO of the present example. FIG. 7 shows the relationship between the measured values of the serum sodium concentration and the predicted values of the serum sodium concentration by the serum sodium concentration prediction model MO, for 133 observation points. As shown in FIG. 7, the observation points are roughly distributed in the vicinity of a straight line indicating perfect prediction, and it can be said the prediction accuracy was very high. The results shown in FIG. 7 are obtained by using linear support vector regression as a learning model and by performing 10-division cross-validation as a validation method.

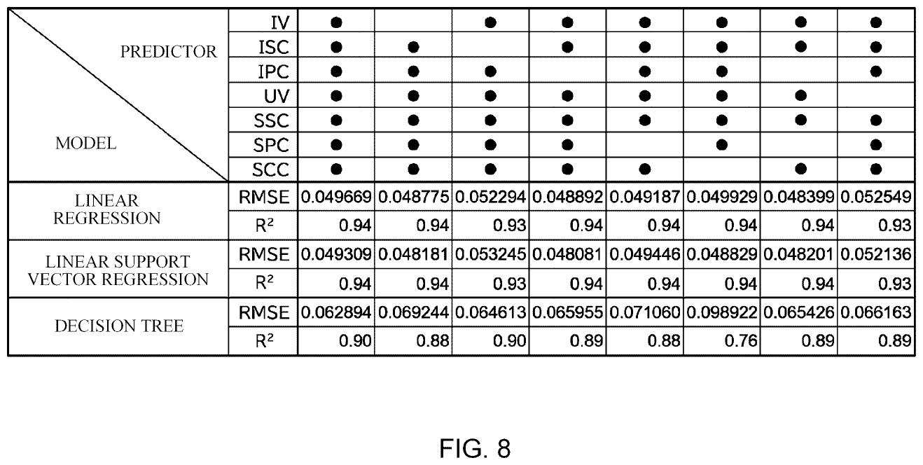

FIG. 8 is an explanatory diagram that illustrates prediction accuracy of the serum sodium concentration prediction model MO when a learning model and a predictor are changed in various ways. FIG. 8 shows the values of two evaluation indices (RMSE and R2) representing the prediction accuracy of the serum sodium concentration prediction model MO for each combination of the learning model and the predictor. Three choices of learning models are set: (1) linear regression, (2) linear support vector regression, and (3) decision tree. Moreover, eight combinations selected from the seven items of the predictors described above are set as the choices for the predictors. In FIG. 8, the seven items of the predictor are indicated by signs (see FIG. 1). In FIG. 8, the items marked with black circles are items that are adopted as predictors, and items not marked with black circles are items that are not adopted as predictors.

As shown in FIG. 8, each combination of the learning model and the predictor show generally high prediction accuracy. For the learning model, the prediction accuracy is especially high when the linear regression and the linear support vector regression are adopted. As for the predictors, the prediction accuracy is highest when all seven items are adopted as predictors as in the above embodiment, but the decrease in prediction accuracy is minimal even when any one or two of the seven items are omitted. Therefore, even when the composition of the predictors is slightly different due to the different equipment and operation of each medical institution, it can be said that the serum sodium concentration can be predicted with high accuracy by using the serum sodium concentration prediction model MO.

### A-6. Effects of the Present Embodiment

As described above, the terminal device 100 of the present embodiment is an information processing device for predicting the serum sodium concentration at a future time (t+Δt) and has the predictor acquisition unit 114 and the prediction execution unit 115. The predictor acquisition unit 114 acquires the predictor, including the measured value of the serum sodium concentration SSC (t) at a reference time (t), and the index value, indicating the the target person's sodium intake in the future period T(0), which is the period from the reference time (t) to the future time (t+Δt), to be predicted. The prediction execution unit 115 inputs the target person's predictor acquired to the serum sodium concentration prediction model MO generated by machine learning using training data TD where the predictor and the measured value of the serum sodium concentration SSC at a future time (t+Δt) are associated with each other, thereby predicting the target person's serum sodium concentration SSC (t+Δt) at the future time (t+Δt).

As described above, the terminal device 100 of the present embodiment can predict the target person's serum sodium concentration SSC (t+Δt) at a future time (t+Δt) with high accuracy by inputting, to the serum sodium concentration prediction model MO, the prediction factors, including the measured value of the serum sodium concentration SSC (t) at the reference time (t) and an index value indicating the target person's sodium intake in the future period T(0) to be predicted. For this reason, according to the terminal device 100 of the present embodiment, it is possible to predict the target person's serum sodium concentration SSC (t+Δt) to be predicted at the future time (t+Δt) with high accuracy only by acquiring the above target person's predictors. Therefore, for example, the medical worker P1 can determine the type and/or the dosage of the infusion so that hyponatremia can be treated as soon as possible while avoiding ODS being caused. It also enables the optimization of room admission based on the prediction of ICU treatment duration.

The terminal device 100 of the present embodiment further has the prediction result output unit 116 for outputting the prediction result of the target person's serum sodium concentration. Therefore, according to the terminal device 100 of the present embodiment, it is possible to make the user of the device recognize the predicted value of the target person's serum sodium concentration SSC (t+Δt) at the future time (t+Δt).

The terminal device 100 of the present embodiment further has the training data acquisition unit 112 for acquiring the training data TD and the model acquisition unit 113 for creating the serum sodium concentration prediction model MO by machine learning using the training data TD. Therefore, according to the terminal device 100 of the present embodiment, the serum sodium concentration prediction model MO can be acquired without using any other device, and the prediction of the target person's serum sodium concentration SSC (t+Δt) at a future time (t+Δt) can be performed using the model.

The terminal device (100) of the present embodiment further has the model updating unit 117 for updating the serum sodium concentration prediction model MO by machine learning using updated training data TD, including data where the target person's predictor and the measured value of the serum sodium concentration SSC (t+Δt) at the future time (t+Δt) are associated with each other. Therefore, according to the terminal device 100 of the present embodiment, the serum sodium concentration prediction model MO can be made a model more suitable for the target person's characteristics, and the prediction accuracy of the serum sodium concentration can be improved.

In the terminal device 100 of the present embodiment, the predictor acquisition unit 114 acquires information for specifying the type of intake items (infusion and/or drinking water) that the target person consumes, and acquires an index value indicating the sodium intake in the future period T(0) by referring to the information (infusion information ID) indicating the sodium content contained in each of intake item. Therefore, according to the terminal device 100 of the present embodiment, it is possible to acquire the index value representing the sodium intake in the future period T(0) only by the user designating the type of the intake item (infusion), and it is possible to more efficiently predict the serum sodium concentration.

In the terminal device 100 of the present embodiment, the predictor further includes the index value representing the water intake amount in the future period T(0) and the index value representing the water discharge amount in the past period T(−1), which is the period from the past time (t−Δt) to the reference time (t). Therefore, according to the terminal device 100 of the present embodiment, the prediction accuracy of the serum sodium concentration can be improved.

In the terminal device 100 of the present embodiment, the target person subject to be predicted is the patient with hyponatremia. Therefore, according to the terminal device 100 of the present embodiment, it is possible to predict the target person's serum sodium concentration when a predetermined treatment is given to a patient with hyponatremia.

The terminal device 100 of the present embodiment further has the notification unit 118 that executes a given notification when the rate of increase in the target person's serum sodium concentration SSC (t) of the target person at a future time (t+Δt) from the measured value of the target person's serum sodium concentration SSC (t) at a reference time (t) to the predicted value thereof is faster than a preset threshold value.

Therefore, according to the terminal device 100 of the present embodiment, in the treatment of the patient with hyponatremia, it is possible to suppress the rate of increase of the serum sodium concentration from becoming excessively fast, and it is possible to avoid the generation of ODS, for example.

### B. Modified Examples

The technique disclosed in the present specification is not limited to the above-described embodiment, and can be modified in various forms without departing from the gist thereof, and for example, the following modifications are also possible.

The configuration of the terminal device 100 in the above embodiment is just an example, and can be modified in various ways. The details of the serum sodium concentration prediction model acquisition process and the serum sodium concentration prediction process in the above embodiment are merely examples, and can be modified in various ways. For example, in the above embodiment, the terminal device 100 acquires the serum sodium concentration prediction model MO by creating the serum sodium concentration prediction model MO, but the terminal device 100 may acquire the serum sodium concentration prediction model MO created by another device (for example, the server 200 in the hospital system 10 or a device on an external network). In this case, the terminal device 100 does not need to include the training data acquisition unit 112.

In the above embodiment, the serum sodium concentration prediction model MO (S330 in FIG. 6) is updated, but updating the serum sodium concentration prediction model MO is not required. In this case, the terminal device 100 does not need to include the model updating unit 117.

In the above embodiment, the notification process (S350 in FIG. 6) is executed, but the notification process does not need to be executed. In this case, the terminal device 100 does not need to include the notification unit 118.

The predictors (FIG. 1) used for creating the serum sodium concentration prediction model MO in the above embodiment are merely examples, and can be modified in various ways. For example, in the above embodiment, the predictor includes the measured value of the serum potassium concentration SPC (t) at the reference time (t), the measured value of the serum chloride concentration SCC (t) at the reference time (t), and the potassium content IPC contained in the infusion, but the predictor does not need to include at least one of these measured values. In the above embodiment, the urine volume UV is used as the index value representing the amount of water discharged in the past period T(−1), but other index values (for example, a body weight change amount, a plasma osmotic pressure, a blood sugar level, and the like) may be used instead of or in addition to the urine volume UV. In the above embodiment, the infusion dose IV and/or the sodium content ISC in the infusion in the future period T(0) is used as the index value representing the sodium intake in the future period T(0), but other index values (for example, the intake of orally ingested substances, such as food and/or the sodium content in the substances) may be used instead of or in addition to the specified index value.

In the above embodiment, the terminal device 100 used by the medical worker P1 has the serum sodium concentration prediction processing unit 111 containing the predictor acquisition unit 114 and the prediction execution unit 115, but at least a part of the function of the serum sodium concentration prediction processing unit 111 may be present in another device (for example, the server 200 in the hospital system 10 or a device on an external network) instead of the terminal device 100. For example, at least a part of the function of the serum sodium concentration prediction processing unit 111 may be incorporated as one function of the electronic medical record system. In this manner, the techniques disclosed herein may be used for, for example, doctor-to-doctor telemedicine consultations. In this manner, the terminal device 100 and/or the other device is an example of the information processing device (or information processing system) in the claims of the present invention. The device may also be a device that predicts other body electrolyte concentrations (e.g., potassium concentration) other than the serum sodium concentration and outputs the predicted value of the serum sodium concentration together with the predicted value.

Although the above embodiment illustrates information processing for predicting the serum sodium concentration when a predetermined treatment is given to the patient with hyponatremia, the technique disclosed in the present specification is not limited to this situation, and can be applied to the prediction of the serum sodium concentration in other situations, as well. For example, the techniques disclosed herein may be similarly applied to predict future serum sodium concentrations in a targeted athlete, such as a long-distance runner, following oral supplementation.

In the above embodiment, a part of the configuration realized by hardware may be replaced by software, and conversely, a part of the configuration realized by software may be replaced by hardware.

### REFERENCE SIGNS LIST

10: Hospital System, 100: Terminal Device, 110: Control Unit, 111: Serum Sodium Concentration Prediction Processing Unit, 112: Training Data Acquisition Unit, 113:
Model Acquisition Unit, 114: Predictor Acquisition Unit, 115: Prediction Execution Unit, 116: Prediction Result Output Unit, 117: Model Update Unit, 118: Notification Unit, 120: Storage Unit, 130: Display Unit, 140: Operation Input Unit, 150: Interface Unit, 190: Bus, 200: Server

1. An information processing device for predicting a serum sodium concentration at a future time, comprising one or more processors and one or more memories, the processor programmed to:
acquire a target person's predictors, which include a measured value of the serum sodium concentration at a reference time and an index value indicating the sodium intake in a future period which is a period from the reference time to the future time;
input the target person's acquired predictors into a serum sodium concentration prediction model created by machine learning using training data where the predictors and a measured value of the serum sodium concentration at the future time are associated with each other, and which predicts the target person's serum sodium concentration at the future time.

2. The information processing device according to claim 1, wherein the processor is programmed to
output a prediction result of the target person's serum sodium concentration.

3. The information processing device according to claim 1, wherein the processor is programmed to:
acquire training data; and
create the serum sodium concentration prediction model by machine learning using the training data.

4. The information processing device according to claim 12 wherein the processor is programmed to:
update the serum sodium concentration prediction model by machine learning using updated training data, including data where the target person's predictors and the measured value of the serum sodium concentration at the future time are associated with each other.

5. The information processing device according to claim 1, wherein the processor is programmed to
acquire information specifying a type of an intake item to be taken by the target person, and acquires an index value indicating the sodium intake in the future period by referring to information indicating the sodium content contained in each of the intake items.

6. The information processing device according to claim 1, wherein
the predictors include: an index value representing water intake in the future period; and an index value representing a water discharge amount in a past period, which is a period from a past time to the reference time.

7. The information processing device according to claim 1, wherein
the target person is a patient with hyponatremia.

8. The information processing device according to claim 7, wherein the processor is programmed to
execute a given notification when the rate of increase in the concentration from the measured value of the target person's serum sodium concentration at the reference time reaches the predicted value of the target person's serum sodium concentration at the future time.

9. An information processing method for predicting a serum sodium concentration at a future time by a computer, comprising:
a process of acquiring a target person's predictor to be predicted, where the predictor includes a measured value of the serum sodium concentration at a reference time and an index value indicating the sodium intake amount in a future period, which is a period from the reference time to the future time; and
a process for inputting the target person's acquired predictors into a serum sodium concentration prediction model created by machine learning using training data where the predictors and a measured value of the serum sodium concentration at the future time are associated with each other, and for predicting the target person's serum sodium concentration at the future time.

10. (canceled)
